(19) United States
(12) Patent Application Publication
(10) Pub. No.: US 2009/0263507 A1
(43) Pub. Date: Oct. 22, 2009

(54) BIOLOGICAL MARKERS AND RESPONSE TO TREATMENT FOR PAIN, INFLAMMATION, NEURONAL OR VASCULAR INJURY AND METHODS OF USE

(75) Inventors: Josee ROY, Memphis, TN (US); Toya D. Kimble, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/105,666

(22) Filed: Apr. 18, 2008

(51) Int. Cl. A61K 33/06; A61P 43/00

(52) U.S. Cl. 424/682

(57) ABSTRACT

The invention provides methods and kits for treatment of pain, inflammation, neuronal or vascular injury and the use of biomarkers for the assessment of the biological activity or disease state. In one embodiment, the kit comprises a biomembrane sealing agent, such as PEG, a bioactive agent, such as a magnesium compound, and an assay for measuring a biomarker. The biomarker is measured pre-, mid- and post-treatment, and results of the measurements are compared to evaluate the treatment, treatment regimen and outcomes.

### FIELD OF THE INVENTION

This invention relates to methods and compositions of treating conditions associated with pain, inflammation, neuronal or vascular injury with the aid of biological markers.

### BACKGROUND OF THE INVENTION

Clinical indications affecting the central nervous system such as traumatic brain injury (TBI), spinal cord injury (SCI) and stroke are leading causes of mortality and morbidity in the industrialized world. For example, about 1 million Americans per year are treated for brain injury in emergency rooms. Approximately 5% of TBI patients die and 30% of the survivors are generally left with moderate to severe disabilities that may impair their ability to return to work or live independently. Following neuronal injury, a significant proportion of patients also develop chronically painful conditions.

Pain in general is associated with a myriad of medical conditions and affects millions of Americans. As reported by the American Pain Foundation, over 50 million Americans suffer from chronic pain, including 20% of individuals aged 60 and over who are affected by joint disorders such as arthritis. Furthermore, nearly 25 million Americans experience acute pain due to injuries or surgical procedures each year. In addition to its economic burden, pain has a tremendous effect on the quality of life of affected individuals and is one of the most common causes of disability.

Accordingly, novel improved methods and compositions of treating pain or inflammation are desired to alleviate these debilitating conditions. In addition, the detection of biological markers in body tissues or fluids may provide an assessment of the neuronal or vascular damage involved or whether an individual may be responsive to the particular intervention and thus an appropriate treatment may be selected.

### SUMMARY OF THE INVENTION

Various embodiments fulfill these and other needs by providing novel kits and methods for treatment of conditions associated with pain, inflammation, neuronal or vascular injury, or pathological conditions accompanied by neuronal injury. Specific embodiments provide a method of assessing or determining injury or assessing recovery following treatment or predictive of treatment outcome by the use of biological markers and determining the appropriate type and level of treatment. In one aspect, a kit is provided for treating a pathological condition associated with pain, inflammation, or neuronal injury comprising at least one biomembrane sealing agent, and at least one bioactive agent. In addition, the kit includes an assay for a biomarker. The biomarker may provide the level and type of injury and whether an individual may be responsive to a particular treatment. Periodic measurements of the biomarker may provide the progression or regression of the injury and an opportunity to change parameters of the intervention to improve the treatment modality. In one embodiment, the composition is incapable of forming a gel.

In different embodiments, the at least one biomembrane sealing agent is selected from the group consisting of polyoxyethylenes, polyalkylene glycol, polyethylene glycol or PEG, polyvinyl alcohol, pluronics, poloxamers, methyl cellulose, sodium carboxylmethyl cellulose, hydroxyethyl starch, polyvinyl pyrrolidine, dextrans, poloxamer P-188, and any combinations thereof.

The at least one bioactive agent is selected from the group consisting of at least one magnesium compound, antioxidants, neurotransmitter and receptor modulators, anti-inflammatory agents, anti-apoptotic agents; nootropic and growth agents; modulators of lipid formation and transport; blood flow modulators; electrical stimulation; and any combinations thereof.

In other embodiments, the at least one bioactive compound comprises at least one of inosine, dexanabinol, electrical or magnetic stimulation, CP 101,606, RPR117824, CD11b/CD18 antibody, CD95 Blocker, ATL-146e, CM101, Riluzole, Topiramate, Amantadine, Gacyclidine, BAY-38-7271, S-1749, YM872, IL-1, IL-8 and TNF-alpha blockers, IL-10, DFU, NXY-059, Edaravone, N-tert-butyl-alpha-phenylnitrone, glutathione and derivates, Rho kinase inhibitors, erythropoietin, steroids, statins IGF-1, GDNF, choline or CDP-choline, creatine, AIT-082, Cyclosporine A, FK-506, Minocycline, Triamcinolone, Methylprednisolone or any combination thereof.

In different embodiments, the at least one magnesium compound comprises magnesium sulfate, magnesium chloride, magnesium gluconate, magnesium ATP, or any combination thereof.

In another aspect, a method is provided for the treatment of a pathological condition associated with pain, inflammation, neuronal injury or vascular injury, or pathological conditions accompanied by neuronal injury. The method includes measuring, directly or indirectly, a biomarker before treatment of the pathological condition. The pathological condition is then treated by delivering to a subject in need thereof a therapeutically effective amount of at least one biomembrane sealing agent and a therapeutically effective amount of at least one bioactive agent comprising magnesium. The biomarker is then measured again, and the pre- and post-treatment measurements are compared to assess the treatment.

In certain embodiments the biological markers assess the type, level or severity of neuronal or vascular injury, or pathological condition associated with a primary or secondary neuronal injury

In other embodiments the biological markers may provide an assessment for a proactive treatment linked to surgery or other invasive intervention that affects the neuronal or vascular structure or the cause of pain.

In certain embodiments one or more than one biomarkers are used comprising ions such as magnesium, sodium, calcium, chloride, phosphate or potassium or protein released by damaged cells including tau, C-tau, neurofilaments, membrane or cytoskeleton elements, degradation products or elements that are part of cellular degradation processes, a protein indicator of lost of homeostasis or cell death by necrosis or apoptosis, indicators of neuronal activity including neuronal transmitters and degradation products such as glutamate and degradation product glutamine, indicators of an inflammatory reaction, cellular or humoral immune response or any combinations thereof.

Another embodiment method includes performing a first measurement procedure to measure a biological marker associated with the pathological condition or with a treatment for the pathological condition. Then, in accordance with one or more results from the first measurement procedure, the pathological condition is treated by administering a therapeutically effective amount of a therapeutic compound comprising magnesium as at least one of the active ingredients of the therapeutic compound. In some embodiments the first measurement procedure is used to determine the potential effectiveness of the treatment, such as biocompatibility with the one or more active ingredients used in the treatment.

In some embodiments a second measurement procedure is performed to detect the biomarker associated with the pathological condition, and results from this second measurement procedure are utilized to evaluate treatment of the pathological condition.

Yet another embodiment for treating a pathological condition includes first treating the pathological condition by administering a therapeutically effective amount of a therapeutic compound comprising magnesium as at least one of the active ingredients of the therapeutic compound, and then performing a first measurement procedure to measure a biological marker associated with the pathological condition or the treatment for the pathological condition. A result from the first measurement procedure is then used to evaluate the treatment of the pathological condition.

In certain embodiments the method further includes performing a second measurement procedure to detect the biomarker associated with the pathological condition, and then utilizing the second measurement procedure to further evaluate treatment of the pathological condition. In some embodiments the method further includes changing treatment of the pathological condition in response to the second measurement procedure.

### DETAILED DESCRIPTION OF THE INVENTION

Various aspects provide novel kits and methods for treatment of conditions associated with pain, inflammation, neuronal or vascular injury, or pathological conditions accompanied by neuronal injury. The discovery of a synergistic effect between PEG, a biomembrane sealing agent, and magnesium is highly significant as it can lead to the development of therapeutic formulations with improved efficacy for the treatment of inflammation, painful conditions and neuronal injuries or pathological conditions associated with neuronal injuries.

### DEFINITIONS

To aid in a better understanding of the various embodiments, the following non-limiting definitions are provided:

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the patient.

The term “subject” includes a living or cultured system upon which embodiment methods and/or kits are used. The term includes, without limitation, humans.

The term “practitioner” means a person who practices embodiment methods, kits, and compositions on the subject. The term includes, without limitations, doctors, other medical personnel, and researchers.

The terms “neuropathic pain” and “neural origin pain” refer to pain initiated or caused by a pathological condition of the nervous system, including, without limitation, pathology following chronic or acute insults.

The hallmarks of neuropathic pain are chronic allodynia and hyperalgesia. Accordingly, the term “allodynia” refers to pain resulting from a stimulus that ordinarily does not elicit a painful response.

The term “hyperalgesia” refers to an increased sensitivity to a normally painful stimulus. Primary hyperalgesia affects the immediate area of the injury.

The term “secondary hyperalgesia” or “referred pain” is normally utilized in cases when sensitization has extended to a broader area surrounding the injury.

The term “neuronal injury” refers to an insult to an element of the central or peripheral nervous systems. Neuronal injuries can be derived from a physical (including mechanical, electrical or thermal), ischemic, hemorrhagic, chemical, biological, biochemical or vascular insults. Examples of neuronal injuries include, without limitations, ischemic and hemorrhagic stroke, spinal cord, brain, cranial nerve and peripheral nerve injuries.

The term “bioactive agent” refers to molecules and physical stimuli.

All references to chemical compounds, including without limitation, bioactive agents, biomembrane sealing agents, and markers, include all forms of these chemical compounds (i.e., salts, esters, hydrates, ethanolates, radioisotopes, imaging probes etc.), wherein said forms possess at least partial activities of the respective chemical compounds.

There are two basic forms of physical pain: acute and chronic. Acute pain, for the most part, results from disease, inflammation, or injury to tissues. It is mediated by activation of sensory fibers also known as nociceptive neurons. Nociceptive pain normally disappears after healing, for example in cases of post-traumatic or post-operative pain. Unfortunately, in some individuals, pathological changes occur that increase the sensitivity of the sensory neurons. In those cases, symptomatic pain can become chronic and persists for months or even years after the initial insult.

Neuronal injuries are complex clinical conditions aggravated by a variety of precipitating causes that influence the severity of injury and ultimately influence the course and extent of recovery. A primary insult to a component of the central and/or peripheral nervous system could be of mechanical, chemical, biological or electrical in nature. Following the primary insult, a cascade of biochemical and physiological events takes place that often leads to pathobiological changes that are considered largely responsible for the development of irreversible damages. This autodestructive cascade is known as secondary injury and because it develops over time after the traumatic event it opens a window of opportunity for pharmacological interventions. Various chronic conditions linked to persistent on-going tissue damage due, for example, to inflammatory reactions, autoimmune or vascular diseases or vascular insults, may also lead to secondary injury of neuronal components and symptomatic pain.

There are at least three major classes of events that are determinant in the secondary phase of the traumatic brain injury (TBI) pathology and other neuronal injuries. One of these is membrane damage to cells that managed to survive the first impact. Small changes in membrane integrity and/or cytoskeletal architecture can impair membrane potential, intracellular transport and ATP production leading to cytoskeletal collapse, mitochondrial dysfunction, energetic failure and free radical production resulting in cell death by either apoptotic or necrotic mechanisms. In some instances, the dying cells can release free radicals and catabolic enzymes (proteinase, peptidases, caspases) that may cause damage to the surrounding cells and increase the number of injured cells. Unfortunately, neuronal cells are particularly vulnerable to membrane damage due to their high energetic demand and their unique anatomical structures that require and increase by many folds the challenge of maintaining efficient membrane integrity and intracellular (particularly axonal) transport.

The other class of events that plays a major role in the secondary phase of neuronal injuries is the storm of neurotransmitter release, which by mechanisms globally referred as “excitotoxicity” can increase the vulnerability of neurons to any additional insults in an area that, in size, extends far beyond the area directly affected by the first insult. For example, a marked increase in extracellular glutamate levels is often associated with neuronal insults. Since glutamate is the most prominent excitatory neurotransmitter of the central nervous system, almost all neurons have glutamate receptors and will be affected by the toxic events triggered by excessive amounts of extracellular glutamate. Excitotoxicity insults are thought to be largely triggered by excessive inflow of Ca²⁺ through a specific subtype of glutamate receptor, the N-methyl-D-aspartate receptors (NMDAR). High concentrations of intracellular Ca²⁺ can activate catabolic enzymes and production of free radicals that may interfere with the repair mechanisms of the cell or its ability to cope with additional challenges or even precipitate cell death.

The third class of detrimental events is linked to vascular damage and breakdown of the blood-brain barrier. In animal models of TBI and SCI, the blood-brain barrier remains disrupted for many days after injury (Schnell et al., 1999) allowing for extravasation of plasma proteins and invasion of the central nervous system (CNS) by blood and immune cells.

Although the role of inflammation in brain injury and other forms of neuronal injuries remains controversial, proper distribution of nutrients to the nervous tissue cannot be accomplished before the blood-brain barrier and the cerebral vessels are repaired.

Inflammation is the body's normal protective response to conditions that include a tissue necrosis component. Tissue necrosis can be derived from a physical (including mechanical, electrical or thermal), chemical, biological or biochemical insult. Clinical conditions with an inflammatory component include traumatic tissue injury, surgery, degenerative diseases such as arthritis and other joint diseases as well as irritation, hypersensitivity, and auto-immune reactions.

During this natural “defense” process, local increases in blood flow and capillary permeability leads to the accumulation of fluids, proteins and immune cells in the inflamed area. Some of these cells can release chemical mediators of inflammation including histamine, cytokines, bradykinin and prostaglandins that can attract more immune cells at the site of inflammation and/or increase the sensitivity of pain fibers within the affected area. As the body mounts this protective response, the symptoms of inflammation develop. These symptoms include, without limitation, pain, swelling and increased warmth and redness of the skin. The inflammatory response has to be tightly regulated otherwise it may lead to tissue necrosis and development of chronic pain.

Biological Markers

Biological markers or biomarkers are found in bodily tissues and/or fluids and may include from a range of simple molecules to complex structures. For example, biological markers may include ions, amino acids, neurotransmitters, sugars, lipids, carbohydrates, proteins, peptides, enzymes, cytokines, receptors, hormones, steroids, genes, ribonucleotides, etc. or any combination thereof. Other more specific biomarkers include magnesium, calcium, glutamate, glutamine, choline, acetylcholine esterase, tau, c-tau, neuron-specific enolase, ubiquitin and ubiquitin enzymes such as ubiquitin hydrolases, n-acetylaspartate, neuronal filaments, myo-inositol, S100B, interleukins, antibodies against the polymer such as antibodies against PEG. Essentially, a biomarker is characterized as an indicator of a particular bodily or disease state. That is, a biomarker may be an indicator of the state of an organism whether in homeostasis or in a disease state. For example, the biomarkers can indicate the progression of the secondary phase that follows a neuronal injury. Biomarkers are thus generally a measure of the cellular, biochemical, physiological, or molecular state or alterations in such parameters in an individual's tissues, cells or fluids.

In addition to measuring normal biological or pathogenic processes of an organism, biological markers are also an objective measure of physiological or biological responses due to therapeutic interventions. Periodic measurements of the biomarkers over time can provide a biological basis for an assessment of the state or progression of the disease condition during therapeutic interventions. A change in the structure, activity and/or level of the selected biomarkers can be indicative of the nature of the primary or secondary injury. Such biomarkers may determine the location, complexity, or severity of the injury. The biomarkers may also assess an individual's susceptibility to certain reactions or treatments. That is, a suitably selected biomarker may assist in identifying individuals that may or may not show the expected response or responses to a particular treatment.

Biomarker information as to their activity levels can also be used to adjust one or more components of the treatment. That is, changes in concentrations of the active ingredients and excipients, dose, dosage regimen, and rate of delivery to yield certain values of the pharmacokinetic parameters, and so forth. The biomarker information can assist in the adjustment of the treatment components on an individual basis, or during clinical trials can provide data to modify the group treatment protocol. Biomarkers can provide an objective and quantifiable measure of the extent of recovery at different time points following treatment.

The detection and evaluation of biomarkers can be based on various imaging techniques, in vivo, in vitro technologies (such as sampling of bodily tissues or fluids and assessing various chemical or biological components or structures) or various combinations thereof. These technologies may detect the biomarker(s) directly, or may detect molecule(s) that interact with the biomarker to reveal and/or amplify the biomarker signal. The use and assessment of one or more biomarkers can further be used to facilitate the development of a new treatment for neuronal injury, such as a neuroprotective therapy like magnesium in a polymer solution for the treatment of spinal cord injury, brain injury, stroke, or peripheral injuries. These biomarkers may also be used to determine the basis for proactive treatment. That is, biomarkers may be used to determine the basis for treatment that is linked to a surgical or other invasive intervention that may affect neuronal or vascular structures or the alleviation of pain or inflammation.

Biomarkers may also be categorized such that they form a continuum for identifying sequences of events (physiological, cellular, biochemical or molecular) that may occur before exposure, or during exposure to an agent and to the disease process. In addition, a category of biomarkers may also identify a sequence of events that reflects the pharmacological effective dosage levels and early responses.

Biomarkers can also identify target tissues that have been pathologically altered through exposures of various pathogens. Biomarker levels on non-target (surrogate) tissues and fluids may also be used to estimate the biomarker levels in the target tissues. The sensitivity, specificity, and reliability or predictability of specific biomarkers and their assays will partly determine the selection criteria.

Moreover, specific markers may be introduced into the body before or during an intervention to monitor the target tissues. The status of the target tissues can be assessed by monitoring the specific markers introduced or monitoring tissue or fluid biomarkers responding to the intervention.

For example, the detection of biomarkers, such as total tau protein, β-amyloid 40 and 42 peptides, are closely associated with some of the typical neuropathological changes that occur in Alzheimer's disease. While prostate specific antigen (PSA) is considered a biomarker for prostate cancer.

Biomembrane Sealing Agents

For more than 40 years, biomembrane sealing agents of various molecular weights have been utilized as adjuncts to culture media for their ability to protect cells against fluid-mechanical injuries. These agents include hydrophilic polymers such as polyoxyethylenes, polyalkylene glycol, polyethylene glycols (PEG), polyvinyl alcohol, amphipatic polymers such as pluronics or poloxamers, including poloxamer P-188 (also known as CRL-5861, available from CytRx Corp., Los Angeles, Calif.) (Michaels and Papoutsakis, 1991) as well as methyl cellulose (Kuchler et al., 1960), sodium carboxylmethyl cellulose, hydroxyethyl starch, polyvinyl pyrrolidine and dextrans (Mizrahi and Moore, 1970; Mizrahi, 1975; Mizrahi, 1983).

Some biomembrane sealing agents including hydroxyethyl starch (Badet et al., 2005) and PEG (Faure et al., 2002; Hauet et al., 2001) have shown effective cryopreservative abilities in organ transplantation studies. Poloxamer P-188 was shown to protect articular cells from secondary injury following mechanical trauma to knee joint which could lead to acute pain and inflammation and potentially develop into a more chronic condition known as osteoarthritis (Phillips and Haut, 2004). Poloxamer P-188 and a neutral dextran protected muscle cells against electroporation or thermally driven cell membrane permeabilization (Lee et al., 1992). Direct application of PEG was shown to anatomically and functionally reconnect transected or crushed axon (Bittner et al., 1986), peripheral nerve (Donaldson et al., 2002) and spinal cord preparations in vitro (Lore et al., 1999; Shi et al., 1999; Shi and Borgens, 1999; Shi and Borgens, 2000; Luo et al., 2002) or in vivo (Borgens et al., 2002). Intravenous or subcutaneous administration of PEG or Poloxamer P-188 improved the cutaneous trunchi muscle reflex response after experimental spinal cord contusion in guinea pigs (Borgens and Bohnert, 2001; Borgens et al., 2004) and improved functional recovery in a naturally occurring spinal cord injury model in dogs (Laverty et al., 2004). PEGs of various molecular weights from 1, 400-20000Da, having a linear or multiple arm structure were shown to improve recovery following tissue injury (Hauet et al., 2001; Detloff et al., 2005; Shi et al., 1999).

Biomembrane sealing agents can be effective following different modes of delivery including local and prolonged cellular exposure, direct and short-term tissue or organ exposure or systemic administration. Effective concentrations of biomembrane fusion agents may vary depending on the purpose and/or mode of delivery. For example, about 0.05% concentration is effective in tissue culture applications (Michaels and Papoutsakis, 1991) and about 30% to about 50% concentration is effective for organ preservation and upon in vivo administration in animals (Hauet et al., 2001; Shi et al., 1999; Borgens and Bohnert, 2001; Borgens et al., 2004).

Bioactive Agents

As discussed above, the inventors have found that a combination of a biomembrane sealing agent and a magnesium compound is useful for the treatment of neuronal trauma and painful conditions. Accordingly, in one embodiment of the invention, the at least one bioactive agent comprises a magnesium compound. Magnesium plays an important role in a large diversity of cellular functions. For example, magnesium is required for glycolysis and oxidative phosphorylation which support energy-producing and energy-consuming reactions in cells. Protein synthesis as well as membrane structure and function are also magnesium-dependent. Levels of magnesium will affect neurotransmitter release including glutamate and acetylcholine release. It also regulates the activity of calcium transporters and opening of the non-methyl-D-aspartate (NMDA) glutamate receptors. Magnesium is known to have anti-oxidant anti-apoptosis effects, and to modulate lipid formation and transport. In addition to its cellular effects, magnesium can modulate physiological functions involved in regulation of blood flow and edema development.

During the last decade, a number of studies have reported that brain levels of free magnesium decline following TBI in animal model and in clinical settings. A decrease in brain levels of free magnesium from 40 to 60% has been observed in various TBI animal models including the fluid percussion model (Vink et al., 1991; Headrick et al., 1994), focal impact model (Suzuki et al., 1997) as well as more diffuse models of brain injury (Heath and Vink, 1996; Smith et al., 1998). Furthermore, in the rodent fluid percussion TBI model, a linear correlation was established between changes in brain free magnesium levels, energetic potential (phosphorylation potential) and functional (motor) outcomes (reviewed in Vink and Cemak, 2000). Decreases in magnesium levels have also been reported in experimental spinal cord injury (Vink et al., 1989).

A direct correlation was also established in TBI patients between this biomarker, that is, the levels of magnesium and the level of recovery (Mendez et al., 2005). TBI patients as well as humans suffering from acute ischemic and/or cerebrovascular events are more susceptible to develop a condition called hypomagnesemia where availability of free magnesium is impaired (Polderman et al., 2000). Hypomagnesemia is also associated with increased mortality in patients in general who require the attention of the intensive care unit (Chernow et al., 1989; Rubeiz et al., 1993).

Magnesium supplementation initiated from minutes to hours after onset of CNS trauma showed neuroprotective effects in animal models of TBI (Heath and Vink, 1999; Esen et al., 2003; Vink et al., 2003; Feng et al., 2004 and Turner et al., 2004), spinal cord injury (Suzer et al., 1999; Kaptanoglu et al., 2003) and stroke (Yang et al., 2000; Westermaier et al., 2003 and 2005).

Clinical evaluation of intravenous administration of magnesium sulfate up to 12 hours following stroke onset showed no significant improvement in a multicenter trial involving 2589 patients (Muir et al., 2004). A follow-up study has been initiated to look at the potential effect of an earlier intervention where magnesium sulfate would be administered within 2 hours of stroke onset (Saver et al., 2004). Another clinical trial initiated in 1999 at the University of Washington (Seattle) was evaluating magnesium sulfate therapy for TBI patients and preliminary data were also negative in this trial.

Magnesium supplementation has also been extensively studied in animals and humans for its ability to reduce acute and chronic pain. However, mixed results have been reported from clinical trials evaluating the efficacy of magnesium (alone or in combination) in reducing pain associated with various surgical procedures (Bolcal et al., 2005; Apan et al., 2004; Bathia et al., 2004; McCartney et al., 2004), headache and acute migraine attacks (Cete et al., 2005; Corbo et al., 2001; Bigal et al., 2002), peripheral neuropathies (Brill et al., 2002; Felsby et al., 1996), cancer (Crosby et al., 2000), primary fibromyalgia syndrome (Moulin, 2001; Russel et al., 1995) and chronic limb pain (Tramer and Glynn, 2002). In addition, it appears that magnesium analgesic effects may be of a short duration such as 4 hours or less (Crosby et al., 2000). Magnesium may also induce side effects such as flushing and aching that can reduce its therapeutic window (Tramer and Glynn, 2002). Magnesium supplementation therapies can be achieved by using various salts including magnesium sulfate, chloride, gluconate and magnesium-ATP leading to similar neuroprotective effects in animal models of CNS injury (McIntosh et al., 1989; Izumi et al., 1991; Hoane et al., 2003; Turner et al., 2004; reviewed in Vink and McIntosh, 1990).

The inventors have found that administration of PEG alone or magnesium alone had no effect on the loss of cognitive functions following brain injury whereas cognitive functions or more precisely, the ability to learn a new spatial task, was improved by more than 30% in animals treated with both PEG and magnesium solutions. Combined treatment with PEG and magnesium was also significantly more potent than treatment with either component alone in animal models of SC1 reducing the lesion size by half, improving locomotor recovery and reducing the occurrence of neuropathic pain. In an acute model of tissue inflammation, the combined PEG and magnesium solution was also more effective than PEG or magnesium alone at reducing symptomatic pain. The discovery of a synergistic effect between PEG, a biomembrane sealing agent, and magnesium is highly significant as it can lead to the development of therapeutic formulations with improved efficacy for the treatment of neuronal trauma, inflammatory and painful conditions.

These results suggest that a biomembrane sealing agent, such as, for example, PEG, may also potentiate the beneficial effects of other therapeutic agents. In different embodiments, such bioactive agents include, neurotransmitter and receptor modulators, anti-inflammatory agents, antioxidants, anti-apoptotic agents, nootropic and growth agents; modulators of lipid formation and transport, electrical stimulation, blood flow modulators and any combinations thereof.

Suitable examples of antioxidants include, without limitation, free radical scavengers and chelators enzymes, co-enzymes, spin-trap agents, ion and metal chelators, lipid peroxidation inhibitors such as flavinoids, N-tert-butyl-alpha-phenylnitrone, NXY-059, Edaravone, glutathione and derivates, and any combinations thereof.

Suitable examples of anti-inflammatory agents, include, without limitation, steroids such as methyl prednisolone, Triamcinolone, modulators of an inflammatory cytokine such as IL-10, IL-1, IL-8, TNF-alpha and their receptors, COX inhibitor such as DFU and modulators of immune cell functions such as CD11b/CD18 antibody.

Suitable examples of neurotransmitter and receptor modulators include, without limitations, glutamate receptor modulators, cannabinoid receptors modulators, and any combinations thereof. A person of ordinary skill in the art will appreciate that one of the receptor modulators is a ligand naturally occurring in a subject's body. For example, glutamate receptors modulators include glutamate.

In another embodiment, at least one bioactive agent is a modulator of glutamate transmission, such as (1S,2S)-1-(4-hydroxyphenyl)-2-(4-hydroxy-4-phenylpiperidino)-1-propanol (also known as CP-101,606), Riluzole (Rilutek®), Topiramate, Amantadine, Gacyclidine, BAY-38-7271, S-1749, YM872 and RPR117824.

In another embodiment, at least one bioactive agent is a cannabinoid receptor modulator such as dexanabinol (Pharmos Corporation, Iselin, N.J., USA).

Anti-apoptotic agents include, without limitations, inhibitors of pro-apoptotic signals (e.g., caspases, proteases, kinases, death receptors such as CD-95, modulators of cytochrome C release, inhibitors of mitochondrial pore opening and swelling); modulators of cell cycle; anti-apoptotic compounds (e.g., bcl-2); immunophilins including cyclosporine A, minocycline and Rho kinase modulators, and any combinations thereof. Suitable non-limiting examples of Rho pathway modulators include Cethrin, which is a modified bacterial C3 exoenzyme (available from BioAxone Therapeutics, Inc., Saint-Lauren, Quebec, Canada) and hexahydro-1-(5-isoquinolinylsulfonyl)-1H-1,4-diasepine (also known as Fasudil, available from Asahi Kasei Corp., Tokio, Japan).

Nootropic and growth agents include, without limitation, growth factors; inosine, creatine, choline, CDP-choline, IGF, GDNF, AIT-082, erythropoietin, Fujimycin (IUPAC name [3S-[3R*[E(1S*,3S*,4S*)],4S*,5R*, 8S*,9E,12R*,14R*,15S*,16R*,18S*,19S*,26aR*]]-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12, 18-tetramethyl-8-(2-propenyl)-15, 19-epoxy-3H-pyrido[2,1-c][1,4]oxaazacyclotricosine-1,7,20, 21 (4H,23H)-tetrone, monohydrate, also known as FK-506 and any combinations thereof.

Suitable non-limiting examples of modulators of lipid formation, storage, and release pathways are apolipoprotein; statins; and any combinations thereof.

Suitable non-limiting of blood flow modulators are adenosine receptor modulators such as ATL-146e and agents that modulate new vessel formation such as CM101.

In yet another embodiment, at least one bioactive agent is an electrical or magnetic stimulation. Electrical or magnetic stimulation may be delivered from a site adjacent to the site of a pathological condition (e.g., trauma). For example, if the pathological condition is a spinal injury at the level of C-6, the electrical or magnetic stimulation may be delivered one segment above and one segment below the trauma (i.e., C-5 and C-7).

A person of ordinary skill in the art will appreciate that multiple sources exist for delivering the electrical or magnetic stimulation. In one embodiment, the source is an Oscillating Field Stimulator (OFS) as described, for example in Shapiro, J. Neurosurg. Spine, 2: 3-10 (2005), incorporated herein by reference in its entirety. Briefly, the outside case of the OFS can be made of materials known to be safe for human applications, such as, for example, a fluoropolymer and a silicone sealant. Inside the case are the power block, timing/switching block, current regulation block, and fail-safe device. The power block provides the direct-current power source for the unit involving a single 3.6-V organic lithium battery with a rated capacity of 2400 mAmp/hour. The timing/switching block includes a complementary metal oxide semiconductor 14-stage binary ripple counter device with an onboard oscillator timed for 15-minute intervals along with a single-pole double-throw analog switch. A fail-safe semiconductor chip is programmed to shut down the OFS if the power falls to 2.6 V, if there is a failure to oscillate, or if there are current changes indicative of an internal short circuit. Current regulation can be set by another semiconductor device that delivers 200 μAmp to each pair of electrodes for a total current of 600 μAmp. The electrodes can be made of standard pacemaker cable and a platinum/iridium tip with a 4.72-mm² surface area. A magnet-controlled reed switch can be used to turn the device on or off. When a magnet is on the switch, the device is turned off. When the unit is turned on, it delivers a field of 500 to 600 μV/mm and a current density of 42.4 μAmp/mm² for each electrode.

Thus, in one embodiment the total current of electrical or magnetic stimulation may be between about 400 μAmp and about 700 μAmp, or between about 450 μAmp and about 650 μAmp, or between about 500 μAmp and about 600 μAmp. The current density of the electrical or magnetic stimulation may be between about 30 μAmp/mm² and about 50 μAmp/mm², between about 40 μAmp/mm² and about 45 μAmp/mm², or about 43 μAmp/mm².

In another embodiment, a transcutaneous electrical nerve stimulation (TENS) may be used as the at least one bioactive agent. A person of ordinary skill in the art will appreciate that one advantage of TENS is that it is non-invasive and that the guidelines for TENS are provided, for example, in Resende et al., Eur. J. Pharmacol. 504:217-222 (2004), incorporated herein by reference in its entirety. Conveniently, the practitioner may use equipment which is commercially available, such as, for example, a Neurodyn III apparatus (IBRAMED, Brazil).

If TENS is selected as the at least one bioactive agent of choice, in different embodiments, the electrical stimuli may be released with a frequency of between about 4 Hz and about 130 Hz, wherein a duration of an individual electrical stimulus is between about 60 and about 200 □s, or between about 100 and about 160 □s or between about 125 and 135 □s.

Thus, a combined treatment comprising an administration of a biomembrane sealing agent, such as for example, one of the polymers disclosed above, and at least one bioactive agent, such as, for example, a magnesium compound, has a positive and synergistic effect in reducing the lesion size, improving functional recovery and reducing chronic pain following neuronal trauma as well as reducing acute pain linked to tissue inflammation.

Accordingly, in one aspect, a pharmaceutical composition includes at least one biomembrane sealing agent and at least one bioactive agent. As discussed above, in one embodiment, the at least one active agent comprises at least one magnesium compound.

A person of ordinary skill in the art will undoubtedly recognize that at least one magnesium compound may be just about any molecule providing a source of magnesium ions, such as, for example, a magnesium salt. In a preferred embodiment, the magnesium salt is non-toxic. Suitable non-limiting examples of the at least one magnesium compound include magnesium sulfate, magnesium chloride, magnesium gluconate, magnesium ATP, and any combination thereof.

Therapeutic Formulations

Therapeutic formulations comprising embodiment pharmaceutical compositions can be prepared for storage by mixing the at least one biomembrane sealing agent and the at least one bioactive agent with optional physiologically acceptable carriers, excipients or stabilizers (see, e.g., Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenyl, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and/or metal complexes (e.g., Zn-protein complexes).

The formulations herein may also contain more than one active compound as necessary for the particular indication being treated, preferably those with complementary activities that do not adversely affect each other. Such molecules are suitably present in combination in amounts that are effective for the purpose intended.

The at least one bioactive agent and/or the at least one biomembrane sealing agent may also be entrapped in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may also be prepared. Suitable examples of sustained-release preparations include semipermeable matrices of solid polymers containing the at least one biomembrane sealing agent and/or the at least one bioactive agent, which matrices are in the form of shaped articles, e.g., films, or microcapsule. Examples of sustained-release matrices include, without limitations, polyesters, hydrogels (for example, poly(2-hydroxyethyl-methacrylate), or poly(vinylalcohol)), polylactides (see, e.g., U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. Polymers such as ethylene-vinyl acetate and lactic acid-glycolic acid enable release of molecules for over 100 days.

A person of ordinary skill in the art will undoubtedly appreciate that the at least one biomembrane sealing agent may be included into or used as the semipermeable matrix. In this embodiment, both the at least one biomembrane sealing agent and the at least one bioactive agent are released as the semipermeable matrix degrades.

Further, a person of ordinary skill in the art will recognize that the at least one biomembrane sealing agent and/or the at least one bioactive agent may be implanted into the subject, for example, in forms of a pump or a depot. A suitable non-limiting design of a depot implant is discussed in details in a co-pending U.S. application Ser. No. 11/403,373 entitled Drug Depot Implant Designs And Methods Of Implantation, filed on Apr. 13, 2006.

In another embodiment, the at least one biomembrane sealing agent and/or the at least one bioagent may be administered locally via a catheter positioned at or near a site of the pathological condition, e.g., neuronal injury. In this embodiment, the catheter has a proximal end and a distal end, the proximal end having an opening to deliver the at least one biomembrane sealing agent and/or the at least one bioagent in situ, the distal end being fluidly connected to a pharmaceutical delivery pump. For example, the proximal end of the catheter delivers the at least one biomembrane sealing agent and/or the at least one bioagent within 10 cm of the site of the pathological condition, more particularly, within 5 cm of the site of the pathological condition, and even more particularly, within 1 cm of the site of the pathological condition. The catheter may be positioned via a minimally invasive procedure, such as, for example, by accessing a blood vessel adjacent or supplying blood to the site of the pathological condition.

It would be within the expertise of a person of ordinary skill in the art to recognize that the at least one biomembrane sealing agent and the at least one bioactive agent may be delivered independently of each other. In one non-limiting example, the at least one biomembrane sealing agent may be delivered through an intramuscular injection and the at least one bioactive agent is delivered via an implant. A person of ordinary skill in the art will undoubtedly recognize that a large number of combinations is possible.

A person of ordinary skill in the art will further recognize that in some cases it may be advantageous to ship and store the at least one biomembrane sealing agent and the at least one bioactive agent separately and pre-mix these compounds at a desired time, e.g., one hour prior to administration, or even to administer those compounds without pre-mixing. Accordingly, in another aspect, an embodiment kit comprises at least one biomembrane sealing agent, at least one bioactive agent, and an assay for a biomarker. In one embodiment of the invention, the composition formed by the biomembrane sealing agent and the bioactive agent is incapable of forming a gel.

A person of ordinary skill in the art will further recognize that the kit provides a practitioner with an advantageous flexibility in selecting the ratios of the at least one biomembrane agent and the at least one bioactive agent.

In another aspect, a method of treating a pathological condition is provided, the method comprising measuring a biomarker associated with a pathological condition, delivering to a subject in need thereof a therapeutically effective amount of at least one biomembrane sealing agent and a therapeutically effective amount of at least one bioactive agent, later again measuring the biomarker, and then comparing the results of the measuring procedures to assess the treatment. In different embodiments, the pathological condition is selected from the group consisting of neuronal injury, tissue injury, surgical intervention, inflammation, and any combination thereof.

Examples of suitable pathological conditions include, without limitations, metabolic neuropathies such as diabetic and alcoholic neuropathies, postherpetic neuralgia, trauma to the central nervous system such as stroke, traumatic brain, spinal cord or cauda equine injuries, pain derived from mechanical or biochemical neuronal insults such as carpal tunnel syndrome, phantom limb pain and symptomatic pain associated with degenerative conditions such as multiple sclerosis, arthritis and other joint diseases, persistent symptomatic pain derived from surgical or other invasive interventions as well as chronic pain derived from injury of peripheral neuronal or non-neuronal tissues.

The therapeutically effective amount of at least one biomembrane sealing agent and the therapeutically effective amount of at least one bioactive agent may be delivered independently of each other by an intravenous administration, an intramuscular administration, intrathecal administration, subcutaneous administration, epidural administration, intra-articular administration, parenteral administration, direct application onto or adjacent to a site of the pathological condition, and any combinations thereof. Initiation of individual treatments could be separated by a few hours, e.g., up to about 24 hours, or more preferably, up to about 16 hours, or more preferably of up to about 8 hours, or even more preferably, up to 4 hours. Thus, the at least one biomembrane sealing agent and the at least one bioactive agent may be delivered from independent sources and/or by different methods, or they may be mixed prior to delivery.

A person of ordinary skill in the art will further recognize that certain invasive procedures, such as, for example, brain or spinal cord surgeries, leave a subject with a neuronal injury. Accordingly, in one embodiment of the invention, the at least one biomembrane sealing agent and/or the at least one bioactive agent is delivered to the subject prior to the event triggering the occurrence of the pathological condition. In one non-limiting example, the event is brain surgery and the pathological condition is an injury to CNS neurons.

Biomarkers and Therapeutic Interventions

A procedure for treating a pathological condition may be performed by first identifying the particular biomarkers associated with a particular condition. The biomarker is then measured and quantified. A therapeutic intervention is performed and then the identified biomarker or biomarkers are quantified again to determine the disease progression or recovery phase. The level of the biomarker before the intervention is compared with post-therapeutic levels. A therapeutic intervention may comprise the application of magnesium in a polymer solution via parenteral administration, wherein the magnesium is in a form of a salt comprising one or more of magnesium sulfate, magnesium chloride, magnesium gluconate or magnesium ATP.

The biomarker or biomarkers may be detected from one or more of blood, cerebrospinal fluid, intercellular fluid, cellular or a tissue sample. The tissue sample can comprise neuronal tissue or any other appropriate bodily tissues that provide the selected biomarker or biomarkers that are associated with the specific pathological condition.

In some situations a specific biomarker that assesses the target tissue may be introduced into the body and thus provide an avenue for monitoring the pathological conditions with exogenous substances.

The measurement of a biomarker before the therapeutic intervention is a first measurement procedure and a second measurement procedure comprises detecting at least one of a structure, activity or level of the biomarker or of a molecule that interacts with the biomarker. The first and second measurement procedures are compared and the biomarkers may be quantitatively or qualitatively assessed thus providing a differential measure of the effectiveness or changes in the pathological condition caused by the intervention. Further measurement procedures of the biomarker sequentially performed post intervention will provide additional time relation measurements that yield the progression or regression of the pathological condition. Alternatively, the level of biomarker in a patient can be compared to reference standard values indicators of homeostasis or pathological conditions.

Depending on the biomarker information the treatment or intervention may be changed to improve the management of the pathological condition. A therapeutically effective amount of a therapeutic compound, for example, a magnesium salt, is administered and the biomarkers assessed before and after such intervention.

A kit may comprise a therapeutically effective amount of a therapeutic compound, such as magnesium, a polymer, or a combination thereof, and an assay for measuring a specific biomarker associated with the pathological condition. Moreover, some kits in addition to magnesium may also contain additional active ingredients to facilitate the therapeutic effectiveness of the intervention.

Once one or more biomarkers are identified and selected, an industry standard assay procedure can be used to measure the biomarkers. The assay procedure may be part of the kit for determining the biomarker(s).

By way of example, a patient may exhibit symptoms of, or otherwise be known to be afflicted by, a pathological condition, such as pain, inflammation, a neuronal injury, or a vascular injury. When treatment with the disclosed novel compositions is considered but prior to administration of the novel compositions, the fluid or tissue samples is collected and the first measurement procedure is performed to measure one or more biomarkers associated with the pathological condition. The specific procedures performed for this first measurement may depend upon the type of biomarkers being measured. For example, the first measurement procedure may involve an amplification step of the measured biomarkers such as DNA or RNA amplification or the use of a linker agent such as an antibody, substrate and signaling molecules or an amplification step linked to a specific imaging technique such as nuclear magnetic resonance or magnetic resonance spectroscopy. Or, the measurement procedure may include administering an exogenous biomarker to the patient, which is then subsequently measured. For example, when using positron emission tomography and other imaging techniques, administration of a molecule interacting with the biomarkers for example a substrate, ligand or an antibody is used to reveal the presence and levels of a biomarker associated with a pathological condition.

The biomarkers measured are ideally associated with the pathological condition that is to be treated. In preferred embodiments, for example, the pathological condition may be a neuronal condition, and the biomarker being measured may be released by the injured neuronal tissue at the time of primary or secondary injury or during the recovery period. The one or more biomarker could include magnesium, calcium, glutamate, glutamine, choline, acetylcholine esterase, tau, c-tau, neuron-specific enolase, ubiquitin and ubiquitin enzymes such as ubiquitin hydrolases, n-acetylaspartate, anti-oxidant molecules or enzymes, neuronal filaments, myo-inositol, S100B, interleukins, or antibodies against the polymer such as antibodies against PEG. In other embodiments, the biomarker may be one or more of an ion, an amino acid, a sugar, a lipid, a protein, a peptide, receptor, neurotransmitter a gene or a ribonucleotide. The one or more biomarkers would be selected based on their ability to predict the type of pathological condition, the treatment regimen or the patient that will produce the expected response to the treatment. For example, if the treatment includes administration of a composition comprising magnesium and PEG for the treatment of a moderate to severe neuronal injury, the biomarkers may comprise pre-treatment levels of molecules released by damaged neuronal tissue such as neuronal filaments and magnesium in a biological fluid to confirm the severity of the neuronal injury. Alternatively, pre-treatment levels of a PEG antibody could be added to insure that the patient is not allergic to PEG. These biomarkers are selected based on animal and human studies where specific pre-treatment levels of the biomarkers are associated with positive treatment-related recovery.

The one or more biomarkers evaluated before treatment and after treatment may be different. For example, in order to assess recovery after treatment, indicators of neuronal damage such as neuronal filaments and magnesium levels may be assessed since measure of serum PEG antibodies may not be relevant at that point. The post-treatment assessment can be performed a few minutes to a few hours after administration of the novel compositions to determine if an additional dose of the novel composition is required or if the dose should be modified (i.e. lower or higher doses). The post-treatment assessment can be performed a few hours to a few weeks or months after administration of the novel compositions to assess recovery from neuronal injury and determine the best rehabilitation strategy.

Measurement of the biomarker may be direct, as when detecting the structure, activity or level of the biomarker, or may be indirect, as by detecting a molecule that interacts with the biomarker. Imaging techniques such as magnetic resonance spectroscopy and nuclear magnetic resonance imaging technique can directly reveal certain molecules and ions whereas other imaging techniques such as positron emission tomography and most of the in vitro techniques may rely on a molecule interacting with the biomarkers for example a substrate, ligand or an antibody to reveal the presence and levels of a biomarker associated with a pathological condition. The preferred measurement would rely on an easy-to-use and rapid in vitro detection test such as those used for pregnancy tests. Measurement, both direct and indirect, of the biomarker will typically involve the extraction of a fluid or tissue sample from the patient. For example, one or more of blood, cerebrospinal fluid, intercellular fluid, cell or a tissue sample may be obtained from the patient, which is then analyzed to detect the biomarker. In specific embodiments, the tissue sample may be extracted from the spinal cord, or may be a blood serum sample, which is then analyzed to detect the requisite biomarker. By way of example, an ELISA test may be performed upon the fluid or tissue sample to detect the biomarker.

Once the first measurement procedure has been performed and the results of detecting the biomarker have been obtained, these results may be used, for example, to determine what therapeutic may be administered, or even if any treatment should be performed at all. Results can reveal the presence or absence or level or structural modification of a biomarker usually not seen in patients not affected by a neuronal injury or in patients with a neuronal injury but who may not have the expected response to the described novel compositions. For example, a patient with a minor injury and only normal levels of neurofilaments and magnesium or a patient who has high levels of PEG antibody prior to treatment and may show an allergic response; in both cases the biomarkers indicate that these patients will not have the expected treatment-related response to the novel compositions. In another example, during a dose-escalation study in human patients, biomarker levels pre- and post-treatment are used to determine the optimal dose of the novel compositions described in this application. Normal levels of biomarkers or levels associated with homeostasis and levels of biomarkers associated with a positive or negative response or no response to treatment can be determined during animal studies and human clinical trials. Results from the first measurement procedure may indicate, for example, the dosing and regimen that should be used for the therapy. Or, the results may indicate that treatment with a particular therapeutic would not be beneficial to the patient.

In preferred embodiments, treatment, when performed, involves the administration of a therapeutic compound that includes magnesium as the active ingredient to treat a neuronal condition. The magnesium may be, for example, in the form of a magnesium salt. In other embodiments, the magnesium is in the form of one or more of magnesium sulfate, magnesium chloride, magnesium gluconate, or magnesium ATP. The magnesium is preferably contained in a polymer solution, such as a solution comprising PEG. The biomarker levels are evaluated before the first dose of the magnesium in PEG solution is administered. The biomarker levels are evaluated again after the first dose to decide if the second dose should be modified. In addition, the biomarker levels are evaluated after the administration of the last dose to determine the optimal dose and as a surrogate marker of long term functional recovery.

After the pathological condition has been treated, the second measurement procedure is performed to again measure the biological marker. To determine the optimal dose and treatment regimen, the biomarkers may be evaluated within a period of time equivalent to seven half-lives of the active components of the novel compositions and more preferably within three half-lives. When used as an early indication of long term recovery, the biomarker levels are evaluated within a few weeks to a few months after treatment, preferably within 1-12 weeks and more preferably within 1-6 weeks post-treatment. The second measurement procedure is preferably performed in the same manner as the first measurement procedure. Alternatively, the biomarkers evaluated before and after treatment may vary. For instance, evaluation of PEG antibody serum levels would be of value before treatment initiation to prevent potential allergic response but may not be useful post-treatment. In addition, the biomarkers most useful to determine prior to treatment if the patient may show the predicted response to the treatment maybe different from the biomarkers most useful as early indicators of long term recovery. For example, biomarkers released from damaged neurons but that cannot cross the blood-brain-barrier are going to be measurable in blood only as long as the blood-brain-barrier is disturbed after injury which usually lasts between a few hours to a few days after injury. These biomarkers are very useful to identify central nervous system injury type and severity but may not be useful as indicators of potential functional recovery. On the other hand, biomarkers released by damaged neurons or involved in repair mechanisms may be more useful when measured at a later time point such as 1-12 weeks post-treatment. When a biomarker level prior to treatment is not available specific levels associated with a response to the treatment (i.e. positive, negative or no response) are determined by prior nonclinical and clinical studies. The results of the first and second measurement procedures are then compared to evaluate how the treatment of the patient is progressing.

In certain embodiments, further treatment of the pathological condition may be changed as a result of comparing the results of the first and second measurement procedures. For example, if after a first dose of the novel composition, a patient showed biomarker levels indicating no response to the treatment, then the second dose of the novel composition may be increased. In other embodiments, as part of a dose-escalation paradigm in patients, biomarker levels pre- and post-treatment may indicate that the treatment regimen has to be modified. Also, various embodiments may perform more than simply two measurement procedures. For example, a series of biomarker measurement procedures and respective treatment protocols may be performed to track progression of the pathological condition and its treatment. Each new round of measurement results may be used to track the pathological condition and adjust the treatment protocol accordingly.

It is not necessary that measurement of the biological marker always be performed before the first treatment protocol. On the contrary, in certain embodiments the pathological condition is first treated by administering a magnesium-containing therapeutic, and then the biomarker is subsequently measured. Results of this measurement procedure may be used to evaluate the effectiveness of the treatment. For example, the results may be compared against a known baseline. Further rounds of measuring and treating may be performed to respectively evaluate and treat the pathological condition, in which each new round of treatment may be adjusted based upon results from the previous rounds of biomarker readings.

All patent and non-patent publications cited in this disclosure are incorporated herein in to the extent as if each of those patent and non-patent publications was incorporated herein by reference in its entirety. Further, even though the invention herein has been described with reference to particular examples and embodiments, it is to be understood that these examples and embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A method for treating a pathological condition comprising:
performing a first measurement procedure to measure a biomarker associated with the pathological condition;
treating the pathological condition by administering a therapeutically effective amount of a therapeutic compound comprising magnesium as at least one of the active ingredients of the therapeutic compound;
performing a second measurement procedure to measure the biomarker associated with the pathological condition; and
comparing at least a result of the first measurement procedure with at least a result of the second measurement procedure to evaluate treatment of the pathological condition.

2. The method of claim 1 wherein the therapeutic compound comprises magnesium in a polymer solution.

3. The method of claim 2 wherein the polymer solution comprises PEG.

4. The method of claim 3 wherein the magnesium is in the form of a magnesium salt.

5. The method of claim 1 wherein the therapeutic compound comprises one or more of magnesium sulfate, magnesium chloride, magnesium gluconate, or magnesium ATP.

6. The method of claim 1 wherein the first and second measurement procedures comprise detecting the biomarker in a sample obtained from one or more of blood, cerebrospinal fluid, intercellular fluid, cellular or a tissue sample.

7. The method of claim 1 wherein the biomarker is at least one of magnesium, calcium, glutamate, glutamine, choline, acetylcholine esterase, tau, c-tau, neuron-specific enolase, ubiquitin and ubiquitin hydrolases, n-acetylaspartate, neuronal filaments, myo-inositol, S100B, interleukins, antioxidants, antibodies against the polymer such as antibodies against PEG.

8. The method of claim 1 wherein the biomarker is at least one of an ion, an amino acid, a sugar, a lipid, a protein, a peptide, receptor, neurotransmitter, enzyme, a gene or a ribonucleotide.

9. The method of claim 1 wherein the first measurement procedure or the second measurement procedure comprises detecting at least one of a structure, activity or level of the biomarker or of a molecule that interacts with the biomarker.

10. The method of claim 1 further comprising performing a third measurement procedure to detect the biomarkers associated with the pathological condition, and comparing at least a result of at least the second measurement procedure with at least a result of at least the third measurement procedure to evaluate treatment of the pathological condition.

11. The method of claim 10 further comprising changing treatment of the pathological condition in response to comparing at least the result of at least the second measurement procedure with at least the result of at least the third measurement procedure or a predetermined reference standard.

12. The method of claim 1 wherein treating the pathological condition by administering the therapeutically effective amount of the therapeutic compound comprising magnesium is performed in accordance with at least a result of the first measurement step or a predetermined reference standard.

13. A method for treating a pathological condition comprising:
performing a first measurement procedure to measure a biological marker associated with the pathological condition or a treatment for the pathological condition; and
in accordance with one or more results from the first measurement procedure, treating the pathological condition by administering a therapeutically effective amount of a therapeutic compound comprising magnesium as at least one of the active ingredients of the therapeutic compound.

14. The method of claim 13 wherein the biomarker is at least one of magnesium, calcium, glutamate, glutamine, choline, acetylcholine esterase, tau, c-tau, neuron-specific enolase, ubiquitin and ubiquitin hydrolases, n-acetylaspartate, neuronal filaments, myo-inositol, S100B, interleukins, antioxidants and antibodies against a polymer.

15. The method of claim 13 further comprising performing a second measurement step to detect the biomarker associated with the pathological condition, and utilizing the second measurement step to evaluate treatment of the pathological condition.

16. The method of claim 15 further comprising changing treatment of the pathological condition in response to the second measurement step.

17. A method for treating a pathological condition comprising:
treating the pathological condition by administering a therapeutically effective amount of a therapeutic compound comprising magnesium as at least one of the active ingredients of the therapeutic compound;
performing a first measurement procedure to measure a biological marker associated with the pathological condition or the treatment for the pathological condition; and
utilizing a result from the first measurement procedure to evaluate the treatment of the pathological condition.

18. The method of claim 17 wherein the biomarker is at least one of magnesium, calcium, glutamate, glutamine, choline, acetylcholine esterase, tau, c-tau, neuron-specific enolase, ubiquitin and ubiquitin hydrolases, n-acetylaspartate, neuronal filaments, antioxidants, myo-inositol, S100B, interleukins, and antibodies against a polymer.

19. The method of claim 17 further comprising performing a second measurement step to detect the biomarker associated with the pathological condition, and utilizing the second measurement step to further evaluate treatment of the pathological condition.

20. The method of claim 19 further comprising changing treatment of the pathological condition in response to the second measurement step.
